(19) United States
(12) Patent Application Publication
(10) Pub. No.: US 2023/0000959 A1
(43) Pub. Date: Jan. 5, 2023

(54) Histone Deacytlase 6 Modulation of Titin Protein Mediated Cardiac Tissue Stiffness and Method for Same

(71) Applicant: The Regents of the University of Colorado, a body corporate, Denver, CO (US)

(72) Inventors: Tomothy A. McKinsey, Broomfield, CO (US); Ying-Hsi Alvin Lin, Singapore (SG); Mark Jeong, Denver, CO (US); Kathleen C. Woulfe, Buffalo Creek, CO (US)

(21) Appl. No.: 17/781,664

(22) PCT Filed: Dec. 2, 2020
(86) PCT No.: PCT/US2020/062943

§ 371 Date: Jun. 1, 2022

Related U.S. Application Data
(60) Provisional application No. 62/942,410, filed on Dec. 2, 2019.

(51) Int. Cl. A61K 38/50; A61K 31/325; A61K 31/437; A61K 38/45; A61P 9/04

(52) U.S. Cl. CPC A61K 38/50; A61K 31/325; A61K 31/437; A61K 38/45; A61P 9/04; A61K 48/00

(57) ABSTRACT

Compositions, methods and kits for treating active or passive titin-induced cardiac muscle stiffness by administering an HDAC6 specific inhibitor or an HDAC6 activator.

### BACKGROUND OF THE INVENTION

Titin is the largest known protein that functions as a molecular spring within sarcomeres, which are the basic contractile units of striated muscle. Mutations in the gene encoding titin are the most common cause of genetic heart disease, and elevated and diminished titin compliance are associated with dilated cardiomyopathy (DCM) and heart failure with preserved ejection fraction (HFpEF), respectively. In the present application, parenthetical reference numerals, e.g., “(1),” refer to the corresponding reference numeral in the References section infra.

Heart muscle stiffness is categorized as either active or passive. Active stiffness is dependent on actomyosin crossbridge interactions, whereas passive stiffness is determined by the myocardial extracellular matrix (ECM) and titin (1). Modulation of titin compliance is linked to human cardiac disease (2), with decreased titin-based passive stiffness observed in patients with systolic dysfunction and dilated cardiomyopathy (DCM) (3-7), and titin stiffening associated with heart failure with preserved ejection fraction (HFpEF) in (8-11). Increased compliance of titin in DCM impairs active contraction and sarcomere length-dependent tension generation (the Frank-Starling mechanism) (12, 13), while excessive titin stiffening in HFpEF diminishes ventricular filling during diastole (2, 9, 14). Given the central role of titin in the control of cardiac homeostasis and disease, there is intense interest in developing therapeutic modalities with the capacity to negatively or positively tune titin compliance.

One mechanism by which titin stiffness is controlled physiologically is through differential splicing to yield two adult isoforms with distinct sizes and stiffness. The larger N2BA isoform (−3.3 MDa) is more compliant than the N2B isoform (−3.0 MDa) (15-17), which is generated by the splicing factor RBM20 (18) and altered titin N2BA/N2B expression ratio is associated with certain forms of heart failure (HF) in humans (14). Another mechanism for regulation of titin compliance is through differential phosphorylation, with the spring elements of titin (N2-Bus and PEVK) serving as hotspots for phosphorylation by kinases such as protein kinase A (PKA), protein kinase G (PKG), protein kinase C (PKC), extracellular signal-regulated kinase (ERK), and Ca2+/calmodulin-dependent protein kinase II (CaMKII) (9). From a therapeutic perspective, most effort has focused on restoring titin phosphorylation by stimulating PKG activity via phosphodiesterase 5 (PDE5) inhibition or soluble guanylyl cyclase (sGC) activation as an approach to decrease passive stiffness of the heart to improve relaxation in patients with HFpEF (19, 20). However, initial clinical trials employing these strategies failed to meet their primary endpoints, suggesting that alternative, kinase-independent methods for manipulating titin stiffness as a therapeutic tactic for heart failure with partial ejection fraction should be explored.

### SUMMARY OF THE INVENTION

In accordance with the present invention, it has been found that modulation of cytosolic histone deacetylase (HDAC), specifically HDAC6, mediates titin-induced stiffness in the heart muscle.

Myofibrils obtained from hearts of HDAC6 knockout (KO) mice exhibit dramatically increased resting tension, an effect that is recapitulated by treatment of cultured adult rat ventricular myocytes (ARVMs) with the HDAC6-selective inhibitor, tubastatin A. Conversely, HDAC6 gain-of-function in ARVMs leads to decreased myofibril stiffness, and ex vivo treatment of rat, human and mouse myofibrils with recombinant HDAC6 increases myofibril compliance. The PEVK domain of titin is rich in proline (P), glutamate (E), valine (V) and lysine (K) and has been suggested as being implicated in elasticity of titin folding and unfolding.

In a pre-clinical model that mimics the diastolic dysfunction component of HFpEF, augmented myofibril stiffness in HDAC6 KO mice is coupled to exacerbated cardiac relaxation impairment, illustrating the pathophysiological significance of HDAC6-mediated titin regulation. These findings define a novel role for a deacetylase in the control of sarcomere function and myocardial stiffness and suggest the potential of targeting HDAC6 to manipulate titin compliance as a means to treat heart disease.

It has been previously demonstrated that inhibition of histone deacetylase (HDAC) catalytic activity with the clinical-stage compound givinostat ([6-(diethylaminomethyl) naphthalen-2-yl] methyl N-[4(hydroxycarbamoyl) phenyl] carbamate), also referred to as ITF2357), ameliorates diastolic dysfunction in murine models of hypertension and aging (21). Among the targets of givinostat is HDAC6, a cytosolic enzyme that deacetylates lysine residues of cytoskeletal proteins (22). Here, the inventors have surprising found that HDAC6 regulates titin compliance. Selective HDAC6 inhibition leads to titin stiffening, whereas HDAC6 activation or gain-of-function increases titin compliance. HDAC6 is currently being aggressively pursued as a therapeutic target for multiple indications, including, without limitation, cancer and neurodegenerative diseases, by virtue of its ability to regulate processes such as protein turnover and mitochondrial transport (23). The present disclosure further details therapeutic approaches for manipulating titin stiffness in the context of human HF based on inhibiting or activating HDAC6 activity and/or expression.

### BRIEF DESCRIPTION OF THE DRAWINGS

FIGS. 1A-K illustrate that HDAC6 loss-of-function increases cardiac myofibril stiffness.

FIG. 1A is a schematic representation of an ex vivo myofibril mechanics system. Myofibrils from 6-month-old male mice were evaluated.

FIG. 1B is a graph of myofibril tension (mN/mm2) generation in response to maximal calcium (pCa 4.5).

FIGS. 1C and 1D are graphs of linear (tREL, slow) and exponential (kREL, fast) myofibril relaxation upon removal of calcium (pCa 9.0), respectively.

FIG. 1E is a graph of myofibril resting tension (mN/mm2) at a sarcomere length of 2.0-2.2. For FIGS. 1B to 1E the dots represent data from individual myofibrils. Mean±SEM is shown, *P<0.05 vs wild type (WT) based on unpaired, two-tailed t-test.

FIG. 1F is a graph of myofibril resting tension-to-sarcomere length curves. Data are presented as mean ±SEM, fitted by third-order polynomials, from four animals per group, with 6-8 myofibrils per mouse analyzed.

FIG. 1G is a graph in which myofibrils were treated with the myosin ATPase inhibitor butanedione monoxime (BDM, 50 mM) prior to assessment of resting tension at the given sarcomere lengths.

FIG. 1H is a schematic representation of the adult rat ventricular myocyte (ARVM) experimental protocol.

FIG. 1I is a graph of resting tension-to-sarcomere length curves obtained with myofibrils isolated from ARVMs treated as indicated. Data are presented as mean ±SEM, fitted by third-order polynomials from four separate ARVM preparations per group, with 6-8 myofibrils per preparation analyzed.

FIG. 1J are images taken from indirect immunofluorescence analysis of acetyl-tubulin and total tubulin in ARVMs treated with vehicle, tubastatin A and givinostat (ITF2357), respectively; scale bar=10 μm.

FIG. 1K are images of immunoblot analysis of acetyl-tubulin and total tubulin in whole-cell homogenates from treated ARVMs.

FIGS. 2A-K illustrate that HDAC6 gain-of-function reduces cardiac myofibril stiffness.

FIG. 2A is a schematic representation of HDAC6 with amino acid numbers indicated for the deacetylase 1 domain and the deacetylase 2 domain.

FIG. 2B are indirect immunofluorescence images of ARVMs infected with adenoviruses encoding FLAG (Millipore Sigma, St. Louis, Mo.)-tagged WT HDAC6, HDAC6 harboring two amino acid substitutions that abolish enzymatic activity (H216/611A), and β-Galactosidase (β-Gal) as a negative control. Insets in the overlay images are line scans of fluorescence intensity in the 488 nm and 568 nm channels within the regions of the cells indicated by the white lines (arrows point to white lines). Averages from the three regions are shown and overlapping peaks of fluorescence reveal co-localization of HDAC6 and sarcomeric α-actinin. DAPI fluorescence of nuclei is also shown. Scale bar=10 μm.

FIG. 2C is a schematic representation of the ARVM experiment protocol employing adenoviruses of FIG. 2B.

FIG. 2D is a graph of myofibril resting tension-to-sarcomere length curves from the ARVM experiment protocol illustrated in FIG. 2C. Data are presented as mean ±SEM, fitted by third-order polynomials, from four animals per group, with 6-8 myofibrils per mouse analyzed.

FIG. 2E is a schematic representation of the ex vivo assay protocol employing vehicle, recombinant HDAC6 and HDAC2 as control and myofibrils isolated from rat or human left ventricles (LV).

FIGS. 2F and 2G are graphs of myofibril resting tension-to-sarcomere length curves from the assay protocol illustrated in FIG. 2E. Data are presented as mean ±SEM, fitted by third-order polynomials, from four animals per group, with 6-8 myofibrils per mouse analyzed.

FIG. 2H is a schematic depiction of titin with amino acid numbers indicated.

FIG. 2I is a schematic representation of the ex vivo assay protocol employing myofibrils from WT and PEVK KO mouse LVs treated with vehicle or recombinant HDAC6.

FIGS. 2J and 2K, are graphs of myofibril resting tension-to-sarcomere length curves for testing WT and PEVK KO mice, respectively, from the assay protocol of Fib 2I. Data are presented as mean ±SEM, fitted by third-order polynomials, from four animals per group, with 6-8 myofibrils per mouse analyzed.

FIGS. 3A-E illustrate that HDAC6 reverses PKC-mediated stiffening of human myofibrils.

FIG. 3A is a schematic representation of titin, with the impact of phosphorylation of the N2B and PEVK regions indicated.

FIG. 3B is a schematic representation of the ex vivo assay protocol employing human myofibrils and recombinant forms of PKCa and HDAC6.

FIG. 3C is a graph illustrating myofibril resting tension measurements at physiologic sarcomere length (˜2.19 μm). Mean +SEM is shown, *P<0.05 vs untreated myofibrils based on one-way ANOVA with Tukey's multiple comparisons test.

FIG. 3D is a graph illustrating myofibril resting tension-to-sarcomere length curves. Data are presented as mean ±SEM, fitted by third-order polynomials. Myofibrils from 3 non-failing human hearts were each treated with vehicle, recombinant PKCa or recombinant PKCα followed by recombinant HDAC6. 6-8 myofibrils per treatment were analyzed and averaged per heart.

FIG. 3E is an immunoblot performed with an anti-PKC substrate antibody and solubilized proteins from myofibrils treated as indicated in the protocol illustrated in FIG. 3B.

FIGS. 4A-L illustrate that HDAC6 deletion exacerbates diastolic dysfunction and cardiac myofibril stiffening.

FIG. 4A is a schematic representation of the mouse model experimental protocol testing diastolic dysfunction in WT and HDAC6 KO mice with preserved ejection fraction driven by combined uninephrectomy (UNX) and deoxycorticosterone acetate (DOCA).

FIG. 4B is a graph of serial Doppler echocardiographic measurements of mitral inflow velocity (E/A), which is a parameter of diastolic cardiac function, from the mouse model experimental protocol illustrated in FIG. 4A. Mean +/−SEM values are shown and were compared by two-way ANOVA with Tukey's multiple comparisons test. *P<0.05 vs WT/Sham, #P<0.05 vs WT/UNX+DOCA.

FIG. 4C are representative E/A images taken 2 weeks start of the protocol illustrated in FIG. 4A.

FIG. 4D is a graph of serial echocardiographic measurements of septal mitral annulus velocity (E′/A′), which is an alternative measure of diastolic function to E/A, taken 2 weeks after the start of the protocol illustrated in FIG. 4A. Mean +/−SEM values are shown and were compared by two-way ANOVA with Tukey's multiple comparisons test. *P<0.05 vs WT/Sham, #P<0.05 vs WT/UNX+DOCA.

FIG. 4E are serial representative E′/A′ images after 2 weeks from the start of the protocol illustrated in FIG. 4A.

FIG. 4F is a graph of invasive, catheter-based measurements of LV end diastolic pressure (LVEDP) at the six-week study endpoint. Mean ±SEM values are shown and were compared by two-way ANOVA with Tukey's multiple comparisons test. *P<0.05 vs WT/Sham.

FIG. 4G is a graph of echocardiographic assessment of systolic function as determined by ejection fraction (EF). Mean +/−SEM values are shown and were compared by two-way ANOVA with Tukey's multiple comparisons test. *P<0.05 vs WT/Sham, #P<0.05 vs WT/UNX+DOCA.

FIG. 4H is a graph representing LV-to-tibia length assessment of cardiac hypertrophy upon necropsy. Mean +SEM values are shown and were compared by one-way ANOVA with Tukey's multiple comparisons test. *P<0.05 vs corresponding Sham control.

FIG. 4I is a graph with quantification of interstitial fibrosis by Picrosirius red staining of LV sections post-necropsy. Mean +SEM values are shown and were compared by one-way ANOVA with Tukey's multiple comparisons test. *P<0.05 vs corresponding Sham control.

FIG. 4J is a schematic representation of the 2-week study protocol to assess the impact of HDAC6 deletion on blood pressure and myofibril stiffness in the mouse model of diastolic dysfunction with preserved ejection fraction.

FIG. 4K is a graph of tail cuff measurements of mean systemic pressure (mmHg) from the study protocol illustrated in FIG. 4J. Data are presented as mean ±SEM.

FIG. 4L is a graph of myofibril resting tension-to-sarcomere length curves taken from the study protocol illustrated in FIG. 4J. Data are presented as mean ±SEM, fitted by third-order polynomials, from four animals per group, with 6-8 myofibrils per mouse analyzed.

FIG. 5 is a graph illustrating that HDAC6 deletion does not alter the kinetics of cardiac myofibril contraction. Kinetic parameters of myofibril tension generation, namely kACT, the rate constant of tension generation following Ca2+ activation, and kTR, the rate constant of tension redevelopment following release-restretch, were unaffected by HDAC6 deletion.

FIGS. 6A-D demonstrate that activity of deacetylase domain 2 of HDAC6 is required to reduce cardiac myofibril stiffness.

FIG. 6A is an immunoblot of adult rat ventricular myocytes (ARVMs) were infected with the indicated adenoviruses and whole-cell homogenates with antibodies against the FLAG epitope, acetyl-tubulin and total tubulin.

FIG. 6B is a densitometry quantification of the immunoblot signals in FIG. 6A. Mean ±SEM values are shown and were compared by two-way ANOVA with Tukey's multiple comparisons test. *P<0.05 vs β-Gal control.

FIG. 6C are images of confocal, indirect immunofluorescence data of ARVMs infected with adenoviruses encoding FLAG-tagged versions of HDAC6 harboring an amino acid substitution that abolishes the catalytic activity of deacetylase domain 1 (H216A) or deacetylase domain 2 (H611A). Scale bar=10 μm.

FIG. 6D is a graph of myofibril resting tension-to-sarcomere length curves 6-8 myofibrils per animal, 4 animals per group were analyzed.

FIGS. 7A-F illustrate that HDAC6 deletion does not alter global titin acetylation or titin isoform levels.

FIGS. 7A and 7B are SDS-PAGE immunoblots in which titin was immunoblotted with antibodies against acetyl-lysine as well as an antibody to the titin Z1Z2 element.

FIGS. 7C-7F are graphs of densitometry of immunoblots quantifying acetylation of titin, relative expression of titin N2BA and N2B isoforms, and total titin. T2 represents a cleavage product of titin. Total titin was normalized to total myosin heavy chain (MyHC) levels determined by Coomassie Brilliant Blue gel staining.

FIGS. 8A-B demonstrate that HJDAC6 deletion does not impact PEVK or N2B phosphorylation.

FIG. 8A is an immunoblot of mouse LV homogenates using phosopho-specific antibodies.

FIG. 8B are graphs illustrating the lack of statistically significant effect of HDAC6 deletion between wild type and knock-out mice on phosphorylated serine at positions 26, 170 and 4010, designated P-S26, P-S170 or P-S4010, respectively.

### DETAILED DESCRIPTION OF THE PREFERRED EMBODIMENTS

The terminology used herein is for the purpose of describing example embodiments only and is not intended to be limiting. For purposes of clarity, the following terms used in this patent application will have the following meanings:

The term “about” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges, is intended to mean an approximation of the value, shape or state referenced. For example, where used with a value, the term “about” is intended to include a variance of ±10% from the stated value, e.g., a stated value of 1 will also include the range of values between 0.9 and 1.1.

The term “substantially” is intended to mean a quantity, property, or value that is present to a great or significant extent and less than, more than or equal to totally. For example, substantially vertical may be less than, greater than, or equal to completely vertical.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the recited range, unless otherwise indicated herein, and each separate value is incorporated into the specification as if it were individually recited herein.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, biomedical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the term “therapeutically effective amount” means that amount of HDAC6 inhibitor or a pharmaceutically acceptable salt thereof that is non-toxic but sufficient to elicits the biological or medicinal response in patients. Therefore, a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine.

As used herein, the term “dose” refers to the measured quantity of HDAC6 inhibitor or a pharmaceutically acceptable salt thereof that be administered to patients at one time.

As used herein, pharmacokinetic parameters refer to in vivo characteristics of HDAC6 inhibitor or a pharmaceutically acceptable salt thereof over time. These parameters include plasma concentration (C), as well as Cₘₘ, C_{avg}, Cₘₐₓ and AUC. The term “AUC” is the area under the curve of a graph of the measured plasma concentration of an active agent vs. time, measured from one time point to another time point. The term “AUC” used herein refers the area under the curve of plasma concentration in each dosing interval at steady state (i.e., from time of initial administration of drug to time t, where t is the length of the dosing interval).

As used herein, the term “optionally” means that the subsequently described event or circumstance may or may not occur, and that the description includes instances where said event or circumstance occurs and instances in which it does not.

One embodiment of the present disclosure is a method for treating a patient who is suffering from titin protein mediated cardiac tissue stiffness by administering a therapeutically effective amount of (i) HDAC6 inhibitor or a pharmaceutically acceptable salt thereof, the method comprising: administering a HDAC6 inhibitor or a pharmaceutically acceptable salt thereof. As a non-limiting example, dosages may exceed 1 μM (IC90) concentration per dose. As further examples, a dosage of about 0.1 mg/kg body weight/day to about 1000 mg/kg body weight/day, for example, a dosage of about 1 μg/kg body weight/day to about 1000 m/kg body weight/day, such as a dosage of about 5 μg/kg body weight/day to about 500 m/kg body weight/day can be useful for treatment of a particular condition.

The dosage may, optionally, be titrated down or up after the initial dosing period depending upon the persons response to administration of drug.

Because the therapeutically effective amount of HDAC6 inhibitor may vary depending on the status of patients, the optional step(s) may be carried out or not.

Pharmaceutical compositions can be administered systemically or locally in any manner appropriate to the treatment of a given condition, including orally, parenterally, intrathecally, rectally, nasally, buccally, vaginally, topically, optically, by inhalation spray, or via an implanted reservoir. The term “parenterally” as used herein includes, but is not limited to subcutaneous, intraarterial, intravenous, intramuscular, intrasternal, intrasynovial, intrathecal, intrahepatic, intralesional, and intracranial administration, for example, by injection or infusion.

For treatment of the central nervous system, the pharmaceutical compositions may readily penetrate the blood-brain barrier when peripherally or intraventricularly administered.

The HDAC6 inhibitors useful in the methods of the invention, can be incorporated into pharmaceutical compositions or formulations. Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Supplementary active compounds (e.g., preservatives, antioxidants, antimicrobial agents including biocides and biostats such as antibacterial, antiviral and antifungal agents) can also be incorporated into the compositions. Preservatives and other additives include, for example, antimicrobials, anti-oxidants, chelating agents and inert gases (e.g., nitrogen). Pharmaceutical compositions may therefore include preservatives, antimicrobial agents, anti-oxidants, chelating agents and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, ears, cornea, conjunctiva, skin or dermis). Thus, pharmaceutical compositions include carriers (excipients, diluents, vehicles or filling agents) suitable for administration to any cell, tissue or organ, in vivo, ex vivo (e.g., tissue or organ transplant) or in vitro, by various routes and delivery, locally, regionally or systemically.

Exemplary routes of administration for contact or in vivo delivery that HDAC6 inhibitor can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, intralymphatic.

Formulations suitable for parenteral administration include aqueous and non-aqueous solutions, suspensions or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be presented in unit-dose or multi-dose kits, for example, ampules and vials, and may be stored in a freeze-dried (lyophilized) condition requiring addition of a sterile liquid carrier, for example, water for injections, prior to use.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, pastes, lotions, oils or creams as generally known in the art.

For topical administration, for example, to skin, pharmaceutical compositions typically include ointments, creams, lotions, pastes, gels, sprays, aerosols or oils. Carriers which may be used include Vaseline, lanolin, polyethylene glycols, alcohols, transdermal enhancers, and combinations thereof. An exemplary topical delivery system is a transdermal patch containing an active ingredient.

For oral administration, pharmaceutical compositions include capsules, cachets, lozenges, tablets or troches, as powder or granules. Oral administration formulations also include a solution or a suspension (e.g., aqueous liquid or a non-aqueous liquid; or as an oil-in-water liquid emulsion or a water-in-oil emulsion).

For airway or nasal administration, pharmaceutical compositions can be formulated in a dry powder, such as a fine or a coarse powder, emulsion, colloid, micelle, or other similar particulate form having an average particle size, for example, in the range of 100 to 500 nm that is administered in the manner by inhalation through the airways or nasal passage. Depending on delivery device efficiency, effective dosage levels typically fall in the range of about 0.1 mg to about 100 mg. Appropriate formulations, wherein the carrier is a liquid, for administration, as for example, a nasal spray or as nasal drops, include aqueous or oily solutions of the active ingredient.

For airway or nasal administration, aerosol and spray delivery systems and devices, also referred to as “aerosol generators” and “spray generators,” such as metered dose inhalers (MDI), nebulizers (ultrasonic, electronic and other nebulizers), nasal sprayers and dry powder inhalers can be used. MDIs typically include an actuator, a metering valve, and a container that holds a suspension or solution, propellant, and surfactant (e.g., oleic acid, sorbitan trioleate, lecithin). Activation of the actuator causes a predetermined amount to be dispensed from the container in the form of an aerosol, which is inhaled by the subject. MDIs typically use liquid propellant and typically, MDIs create droplets that are 15 to 30 microns in diameter, optimized to deliver doses of 1 microgram to 10 mg of a therapeutic. Nebulizers are devices that turn medication into a fine mist inhalable by a subject through a face mask that covers the mouth and nose. Nebulizers provide small droplets and high mass output for delivery to upper and lower respiratory airways. Typically, nebulizers create droplets down to about 1 micron in diameter.

Dry-powder inhalers (DPI) can be used to deliver the compounds of the present invention, either alone or in combination with a pharmaceutically acceptable carrier. DPIs deliver active ingredient to airways and lungs while the subject inhales through the device. DPIs typically do not contain propellants or other ingredients, only medication, but may optionally include other components. DPIs are typically breath-activated but may involve air or gas pressure to assist delivery.

For rectal administration, pharmaceutical compositions can be formulated as a suppository with a suitable base comprising, for example, cocoa butter or a salicylate. For vaginal administration, pharmaceutical compositions can be formulated as pessaries, tampons, creams, gels, pastes, foams or spray formulations containing in addition to the active ingredient a carrier, examples of appropriate carriers that are known in the art.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

HDAC6 inhibitor formulations may be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (excipient, diluent, vehicle or filling agent) that, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound. Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact with the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

In the methods of the invention, HDAC6 inhibitors may be administered in accordance with the methods at any frequency as a single bolus or multiple doses e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly or monthly. Timing of contact, administration ex vivo or in vivo delivery can be dictated by the infection, reactivation, pathogenesis, symptom, pathology or adverse side effect to be treated. For example, an amount can be administered to the subject substantially contemporaneously with, or within about 1-60 minutes or hours of the onset of a symptom or adverse side effect of COVID-19 infection, reactivation, or pathogenesis.

Doses may vary depending upon whether the treatment is therapeutic or prophylactic, the onset, progression, severity, frequency, duration, probability of or susceptibility of the symptom, the type of virus infection, reactivation or pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the infection, reactivation, pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

The dosage unit involved depends, for example, on the condition treated, nature of the formulation, nature of the condition, embodiment of the claimed pharmaceutical compositions, mode of administration, and condition and weight of the patient. Dosage levels are typically sufficient to achieve a tissue concentration at the site of action that is at least the same as a concentration that has been shown to be active in vitro, in vivo, or in tissue culture. Exemplary dosages may exceed 1 μM (IC90) concentration per dose. As further examples, a dosage of about 0.1 mg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day can be useful for treatment of a particular condition.

Another aspect of this disclosure provides pharmaceutical kits containing a pharmaceutical composition of this disclosure, prescribing information for the composition, and a container. The HDAC6 inhibitor composition is useful for modulating cardiac muscle stiffness when administered in a kit or container containing a formulation of HDAC6 inhibitor and a pharmaceutically acceptable carrier. Instructions for use of the HDAC6 inhibitor, including, without limitation, the acceptable routes of administration, dosages, and therapeutic or prophylactic administration regimens for treatment or prophylaxis of cardiac muscle stiffness may, optionally, be included with or associated with the container or kit. The container or kit may optionally include dose packs, vials, syringes, needles, dispensers, tubes, bottles, patches, or such other containers for the HDAC6 inhibitor formulation and medical equipment required to dispense and administer the HDAC6 inhibitor formulation to a patient. Similarly, the HDAC6 activator composition is useful for modulating cardiac muscle stiffness when administered in a kit or container containing a formulation of HDAC6 activator and a pharmaceutically acceptable carrier. Instructions for use of the HDAC6 activator, including, without limitation, the acceptable routes of administration, dosages, and therapeutic or prophylactic administration regimens for treatment or prophylaxis of cardiac muscle stiffness may, optionally, be included with or associated with the container or kit. The container or kit may optionally include dose packs, vials, syringes, needles, dispensers, tubes, bottles, patches, or such other containers for the HDAC6 activator formulation and medical equipment required to dispense and administer the HDAC6 activator formulation to a patient.

### MATERIALS AND METHODS

Animal Models

Animal experiments were approved by the Institutional Animal Care and Use Committee at the University of Colorado Denver. Sprague Dawley (SD) rats were obtained from Charles River Laboratories for ARVM isolation. For the diastolic dysfunction model, ten week-old male WT and HDAC6 KO mice, backcrossed to the sv129 S6/SvEvTac background, were subjected to right unilateral nephrectomy (UNX). One week later, mice underwent a subcutaneous implantation of 25 mg deoxycorticosterone acetate (DOCA; Sigma Aldrich D7000) impregnated in a medical grade 100% silastic pellet (approximately 1000 mg/kg) or a sham-vehicle pellet. Following implantation, mice that received the DOCA pellet were given tap water containing 0.9% NaCl and 0.2% KCl to drink.

Hemodynamic Analysis

Serial transthoracic echocardiography and Doppler analyses were performed using a Vevo2100 instrument (Visual Sonics) to simultaneously obtain ECG recordings and assess cardiac dimensions and LV function. Animals were anaesthetized with 2% isoflurane, their chests shaved, and body temperature maintained at 37° C. Long and short parasternal axes views of the LV were obtained. Short axis 2-dimensional views of the LV at the papillary muscle level were used to acquire M-mode targeted recordings. Anterior and posterior end-diastolic and end-systolic LV wall thickness and internal diameter were measured using the leading edge method of the American Society of Echocardiography to calculate ejection fraction (EF). Doppler signals of mitral inflow and myocardial tissue movement at the level of the mitral annulus were obtained to calculate the ratio of early and active filling wave-peak of diastolic flow velocity (E/A), and the ratio of peak diastolic tissue velocity (E′/A′), in order to assess diastolic cardiac function. All measurements were averaged from three consecutive cardiac cycles on the exhale phase. Serial blood pressure measurements were taken in conscious animals using a noninvasive tail-cuff system (CODA, Kent Scientific). To minimize the impact of anxiety on blood pressure readings, mice were taken through the entire procedure for 5 consecutive days prior to obtaining the measurements. Immediately prior to the conclusion of the study, LV end diastolic pressure was measured using an invasive catheter in mice anesthetized with 1.5% isoflurane (Scisense Inc.).

Exercise Testing

Exercise tests were performed on a custom-built motor-driven treadmill (Exer 3/6 Treadmill, Columbus Instruments, Columbus Ohio, USA) following a 5 day acclimatization period in which animals were familiarized with treadmill running for 5 minutes/day at a speed of 10 m/min up a 5% incline. For the last several runs, the speed of the treadmill was increased progressively over the last minute to ˜15-25 m/min to familiarize the mice with high-speed running. Following a brief 2 minute run at 10 m/min and a 1 minute rest (warm-up), mice underwent a series of 4 runs to exhaustion at speeds of 10, 20, 30, and 40 m/min performed at random on separate days for the determination of critical speed (CS). Mice were encouraged to run by applying manual bursts of high-pressure air at the hindlimbs whenever they drifted towards the back of the treadmill lane. Tests were terminated whenever the mice began to show an obvious change in running gait, remained at the rear of the treadmill, or were unable or unwilling to keep pace with the treadmill belt despite obvious exertion of effort. CS was calculated from the slope (a) and the intercept (b) of the regression line, plotting the distance vs. the time to exhaustion from the 4 tests according to the equation y=ax+b, as previously described (46).

Myofibril Mechanics Analyses

Myofibril mechanics were quantified using the fast solution switching technique (47). Frozen LV sections were skinned in 0.5% Triton-X in rigor solution (132 mM NaCl, 5 mM KCl, 1 mM MgCl_{2,} 10 mM Tris, 5 mM EGTA, pH 7.1) containing protease inhibitors (10 μm leupeptin, 5 μm pepstatin, 200 μm PMSF and 10 μm E64), as well as 500 μm NaN₃ and 500 μm DTT at 4° C. overnight. Skinned LVs were washed in fresh rigor solution and homogenized (Tissue-Tearor, Thomas Scientific) in relaxing solution (pCa 9.0) containing protease inhibitors. Myofibril suspensions were transferred to a temperature controlled chamber (15° C.) containing relaxing solution (pCa 9.0; 100 mM Na₂EGTA; 1 M potassium propionate; 100 mM Na₂SO₄; 1 M MOPS; 1 M MgCl₂; 6.7 mM ATP; and 1 mM creatine phosphate; pH 7.0). Myofibril bundles were mounted between two micro-tools. One tool was connected to a motor that could produce rapid length changes (Mad City Labs). The second tool was a calibrated cantilevered force probe (7-10 μm/μN; frequency response 2-5 KHz). Myofibrils were set 5-10% above slack myofibril length. Average sarcomere lengths and myofibril diameters were measured using ImageJ software. Mounted myofibrils were activated and relaxed by rapidly translating the interface between two flowing streams of solutions of different pCa. Data were collected and analyzed using customized LabView software. Measured mechanical and kinetic parameters were defined as follows: resting tension (mN/mm²)=myofibril basal tension in fully relaxing condition; maximal tension (mN/mm²)=maximal tension generated at full calcium activation (pCa 4.5); the rate constant of tension development following maximal calcium activation=kAcT; and relaxation parameters were defined as: duration of the linear relaxation=linear duration, and the rate constant of exponential relaxation=fast k_{REL}. For, myofibril resting tension-to-sarcomere length curves, each myofibril was stretched incrementally to determine the resting tension at different sarcomere lengths, and a resting tension-sarcomere length relationship was measured. The data from all myofibrils of the same animal were grouped into 0.15-μm intervals of sarcomere length and averaged. The averaged data of all animals from each group were shown as mean ±(or +) SEM values fitted by third-order polynomials.

Human hearts were obtained from a tissue bank maintained by the Division of Cardiology at the University of Colorado-Denver (COMIRB 01-568). All patients were followed by the University of Colorado Heart Failure Program and offered participation in the research protocol. Hearts were collected at the time of orthotopic cardiac transplantation.

Ex vivo treatment of myofibrillar proteins with HDAC6 and/or PKCa

Myofibril pellets were washed twice with reaction buffer (25 mM of Tris-HCl, pH 8.0, 137 mM of NaCl, 2.7 mM of KCl, 1 mM of MgCl₂, 0.1 mg/ml BSA). Washed myofibril lysates were resuspended in 200 μL reaction buffer with or without recombinant HDAC6 (1 μg/μL, BPS Bioscience) for 30 minutes at 15° C. The second aliquot was resuspended in reaction buffer only. For the treatment of recombinant PKCa and recombinant HDAC6, myofibril lysates were resuspended in 1504, of relaxing solution containing recombinant PKCα (0.066 U/μL, Millipore 14-484) and lipid activator (Millipore 20-133) for 30 minutes with or without the addition of recombinant HDAC6 (1 μg/μL, BPS Bioscience) for another 30 minutes. After treatment, myofibril lysates were washed twice with relaxing solution containing protease inhibitors and mechanics studies were performed.

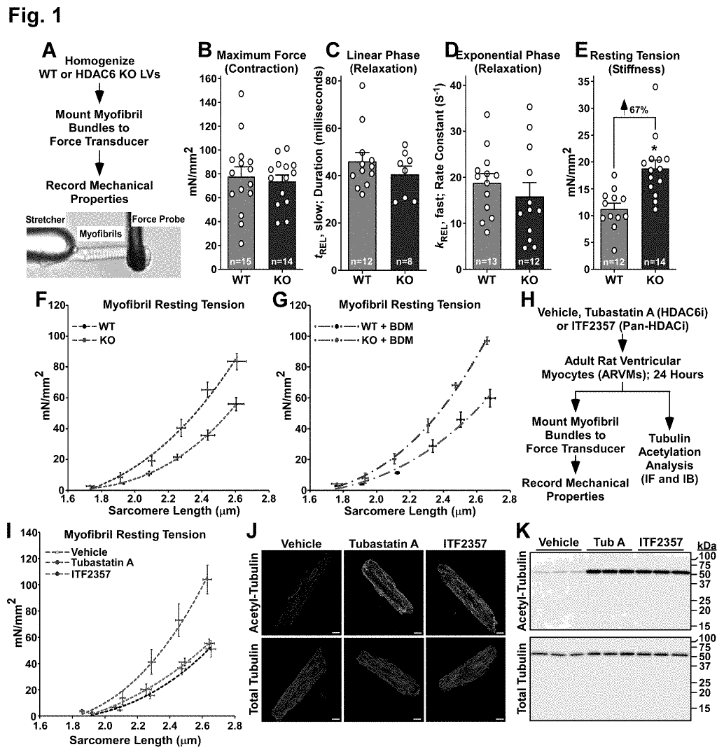

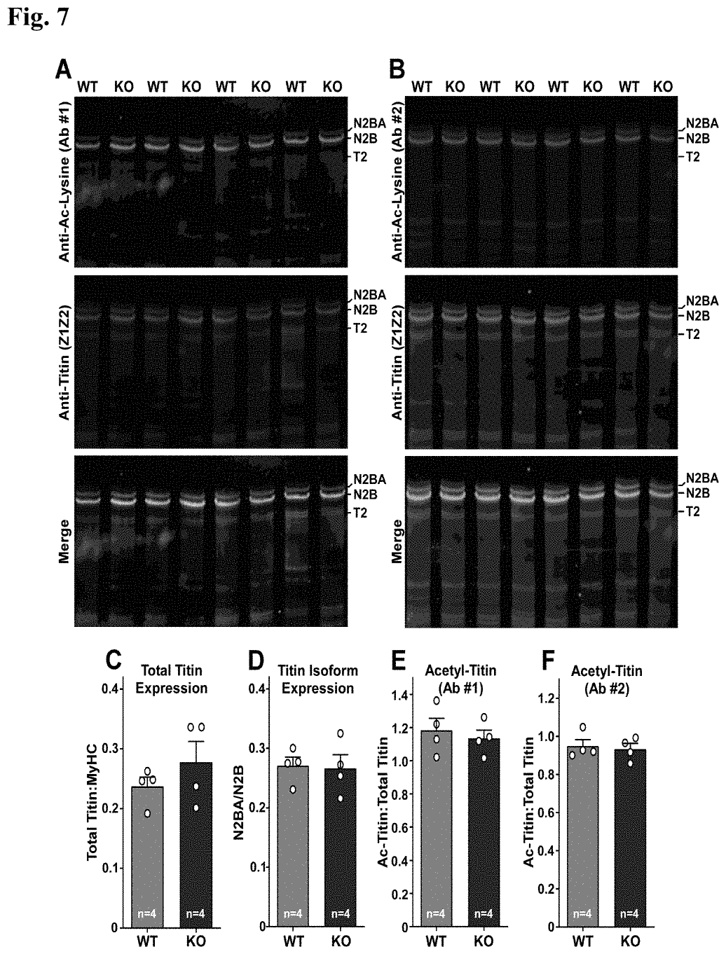

HDAC6 loss-of-function leads to titin stiffening and, hence, cardiac muscle stiffness. To determine whether HDAC6 regulates diastolic function of the heart by augmenting titin stiffness, myofibrils obtained from homogenized left ventricles (LVs) of wild type (WT) versus HDAC6 knockout (KO) mice were mounted on a force transducer in a tissue bath and their mechanical properties were quantified ex vivo (FIG. 1A). Myofibrils from HDAC6-deficient mice generated force in response to Ca2+ and relaxed upon Ca2+ removal equivalently to those from hearts of WT mice (FIG. 1B to D, and FIG. S1). In contrast, myofibril resting tension, which is a surrogate for passive stiffness, was elevated in HDAC6 KO mice compared to WT controls (FIG. 1E). This finding is counter to our hypothesis that inhibition/deletion of this HDAC isoform would reduce myofibril stiffness. Altered myofibril function in HDAC6 KO mice was not associated with changes in global titin isoform expression or global acetylation state of the giant protein (FIGS. 7A-7F).

Myofibrils stretched beyond slack length develop greater passive tension. To evaluate the impact of HDAC6 on cardiac stiffness, myofibrils from WT and KO hearts were subjected to stepwise extension ex vivo, and concomitant alterations in resting tension were quantified to yield sarcomere length-to-resting tension curves. HDAC6 deletion resulted in a profound leftward shift in the sarcomere length-to-resting tension curve compared to WT controls (FIG. 1F). A similar shift was observed employing myofibrils treated with the myosin ATPase inhibitor, butanedione monoxime (BDM) (FIG. 1G), ruling out the possible contribution of residual force-generating cross-bridges to this phenotype, and further suggesting that HDAC6 deletion diminishes titin compliance.

The following example tested whether HDAC6 catalytic activity regulates myofibril stiffness.

Example 1: Cultured adult rat cardiac myocytes (ARVMs) were treated for 24 hours with one of three compounds: 1) tubastatin A, an HDAC6-selective inhibitor, 2) givinostat (ITF2357), a non-selective HDAC6 inhibitor that inhibits HDAC6 as well as several other zinc-dependent HDACs, or 3) vehicle control (FIG. 1H). Consistent with the findings from KO hearts, myofibrils from ARVMs exposed to tubastatin A exhibited elevated resting tension (FIG. 1G). Surprisingly, givinostat had no effect of myofibril stiffness. Pharmacodynamic assessment of tubulin acetylation as a marker of HDAC6 inhibition confirmed that tubastatin A and givinostat inhibited HDAC6 equivalently (FIGS. 1H and 1I). Thus, enhanced titin stiffness is only observed upon selective inhibition of HDAC6, and concomitant targeting of other HDAC isoform(s) by givinostat appears to override this effect. These findings are in agreement with the prior demonstration that givinostat ameliorates rather than exacerbates diastolic dysfunction in murine models (21).

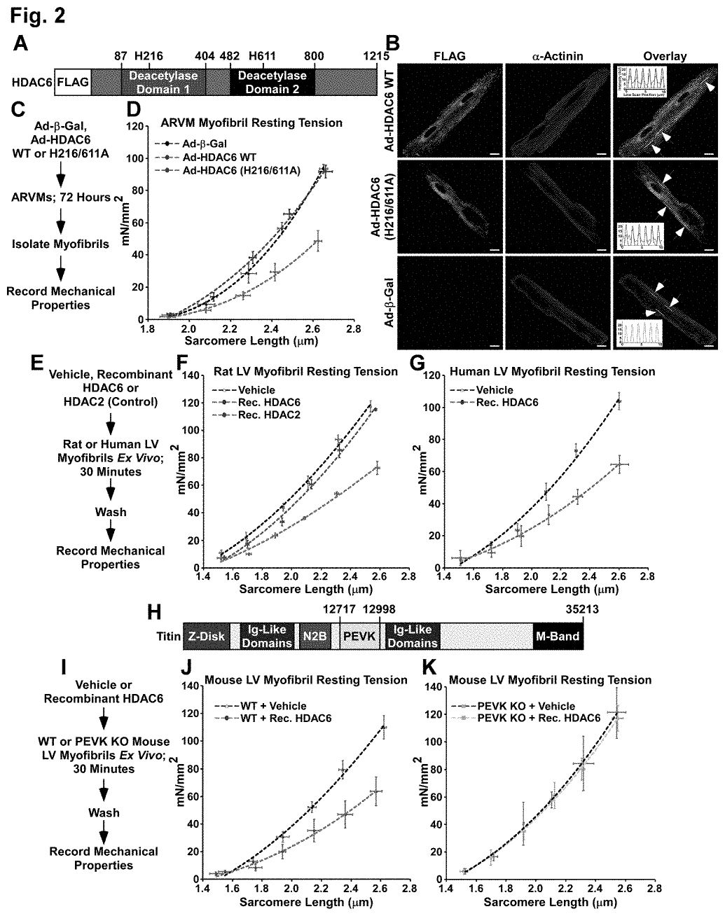

Unlike other HDACs, HDAC6 contains tandem deacetylase domains (FIG. 2A). Deacetylase domain 1 extends from amino acid 87 to amino acid 404 and includes histidine at position 216 (H216). Deacetylase domain 2 extends from amino acid 482 to amino acid 800 and includes histidine at position 611 (H611). The following example tested whether HDAC6 gain-of-function or activation leads to titin-mediated cardiac muscle stiffness.

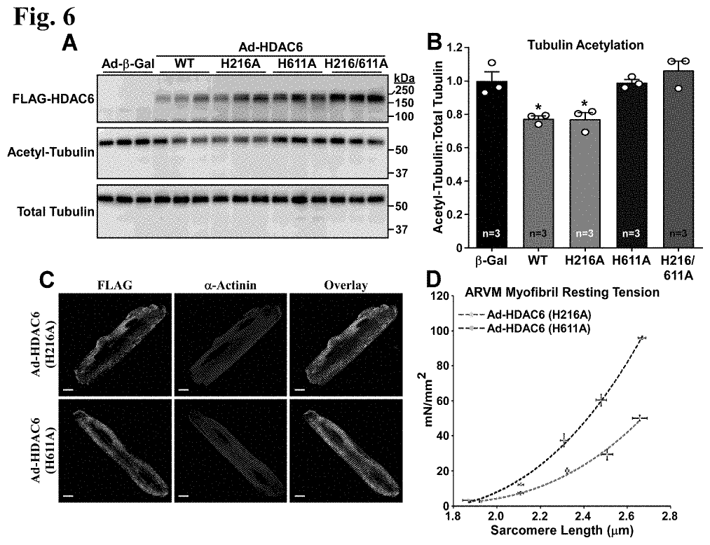

Example 2: Cultured adult rate cardiac myocytes (ARVMs) were infected with adenoviruses expressing wildtype HDAC6 (Ad-HDAC6 WT), derivatives of the enzyme harboring specific histidine amino acid substitutions that abolish catalytic activity at positions 216, 611 or both 216 and 611 (Ad-HDAC6 H216A, H611A or H216/611A), or a β-galactosidase negative control (Ad-β-Gal); immunoblotting confirmed efficient expression of ectopic HDAC6 in the cultured adult cardiomyocytes, and the ability of the deacetylase domain 2 to govern deacetylation of α-tubulin (FIGS. 6A-6B) (24, 25). Consistent with a role for HDAC6 in controlling myofibril function, confocal imaging revealed a pool of the enzyme that co-localized with sarcomeric a-actinin at the Z-line in cardiomyocytes independently of its catalytic activity (FIG. 2B and FIG. 6C).

To determine whether HDAC6 gain-of-function alters passive stiffness of cardiomyocytes, ARVMs were infected with the aforementioned adenoviruses for 72 hours and myofibrils were subsequently isolated for evaluation of mechanics (FIG. 2C). Strikingly, ectopic expression of HDAC6 dramatically increased the compliance of cardiac myofibrils in a manner dependent on the catalytic activity of deacetylase domain 2 (FIG. 2D, and FIG. 6D). Furthermore, ex vivo incubation of purified rat myofibrils with recombinant HDAC6, but not HDAC2, reduced titin stiffness (FIGS. 2E-2F), establishing the ability of HDAC6 to directly modulate sarcomere function.

Example 3: Wild type or PEVK knock out mouse left ventricle myofibrils were exposed ex vivo to recombinant HDAC6 (rHDAC6) or vehicle for control for 30 minutes. The protocol is outlined in FIG. 2I. After washing, the mouse myofibrils were tested for mechanical properties and it was found that rHDAC6 also increased compliance the mouse myofibrils.

Example 4: Human myofibrils were obtained from non-failing donor left ventricle explants and exposed to the same test protocol with rHDAC6 as in Example 3. Mechanical testing of the rHDAC6-treated human myofibrils indicated that demonstrated a conserved ability of this deacetylase to control cardiomyocyte passive stiffness in higher mammals (FIG. 2G).

The modular structure of titin, which consists of greater than 30,000 amino acids, is depicted (FIG. 2H). To begin to define the deacetylase-responsive region(s) of titin, recombinant HDAC6 was incubated with myofibrils obtained from adult mouse hearts from WT mice or mice in which the PEVK region of titin was deleted by homologous recombination (FIG. 2I). As with samples obtained from rat and human hearts, HDAC6 efficiently reduced the compliance of mouse cardiac myofibrils (FIG. 2J). Myofibrils from mice lacking the PEVK region of titin were completely resistant to HDAC6 (FIG. 2K), thus suggesting a role of the PEVK domain in in HDAC6-mediated regulation of cardiomyocyte passive stiffness.

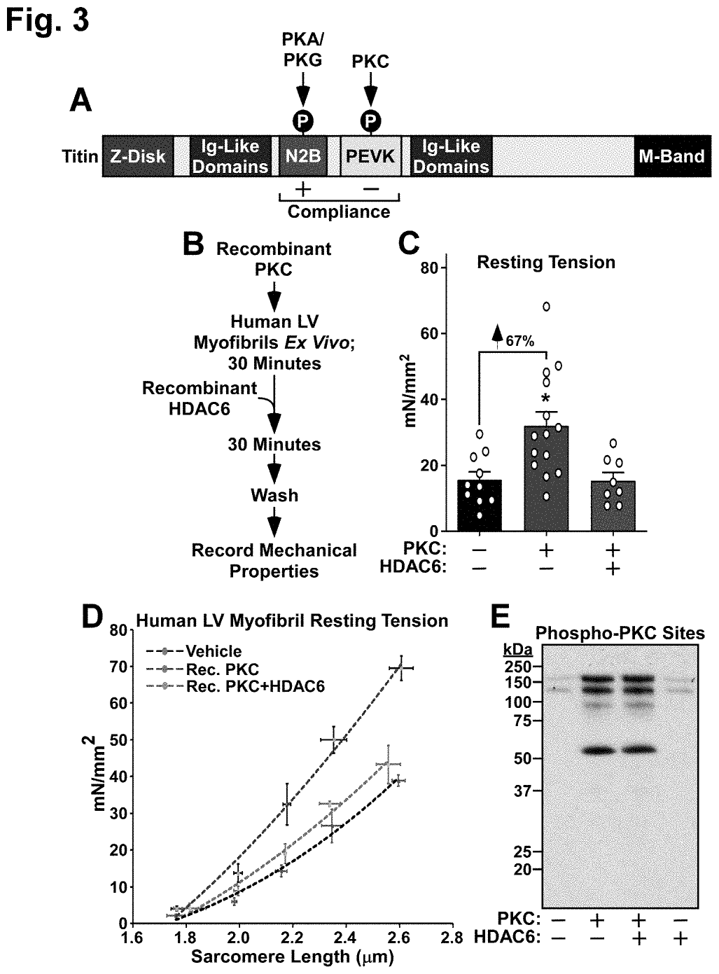

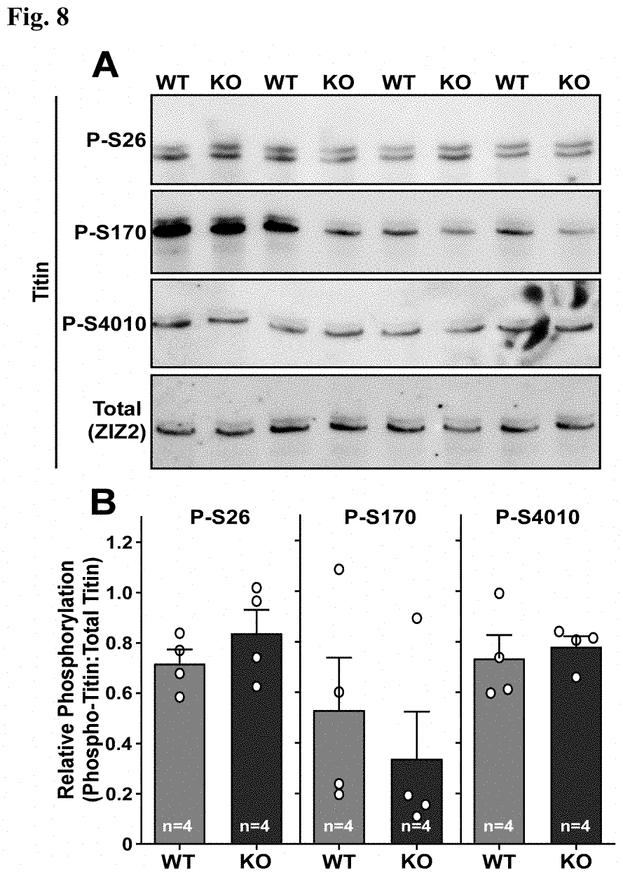

HDAC6 reverses PKC-mediated titin stiffening in human myofibrils. PKC-dependent phosphorylation of the PEVK element of titin leads to myofibril stiffening, while PKA/PKG-mediated phosphorylation of the adjacent N2B region increases titin compliance (FIG. 3A). Immunoblotting of mouse LV homogenates using phospho-specific antibodies failed to reveal an impact on HDAC6 deletion on PEVK or N2B phosphorylation (FIGS. 8A-8B), suggesting that HDAC6 regulates titin stiffness by directly deacetylating the protein as opposed to indirectly affecting its phosphorylation state.

Elevated PEVK phosphorylation is associated with increased cardiomyocyte passive stiffness in pre-clinical models and in human heart failure (7). To begin to address the therapeutic potential of HDAC6 gain-of-function, an ex vivo experiment was performed to determine whether HDAC6 is capable of overriding the cardiac stiffening effect of PKC. Myofibrils obtained from non-failing human LVs were pretreated with recombinant PKCα, washed, and then incubated with recombinant HDAC6 (FIG. 3B). PKCα dramatically increased myofibril resting tension at physiologic sarcomere length and, remarkably, this stiffening was completely normalized upon subsequent exposure of the myofibrils to HDAC6 (FIG. 3C). Recombinant HDAC6 also largely blocked the PKC-induced leftward shift in the sarcomere length-to-resting tension to curve, without affecting PKC-driven phosphorylation of sacromeric proteins (FIGS. 3D-3E). These data demonstrate the ability of HDAC6 to neutralize PKC-mediated stiffening of human myofibrils through a mechanism that is independent of phosphorylation, suggesting that HDAC6 gain-of-function could be therapeutically beneficial in the setting of HFpEF.

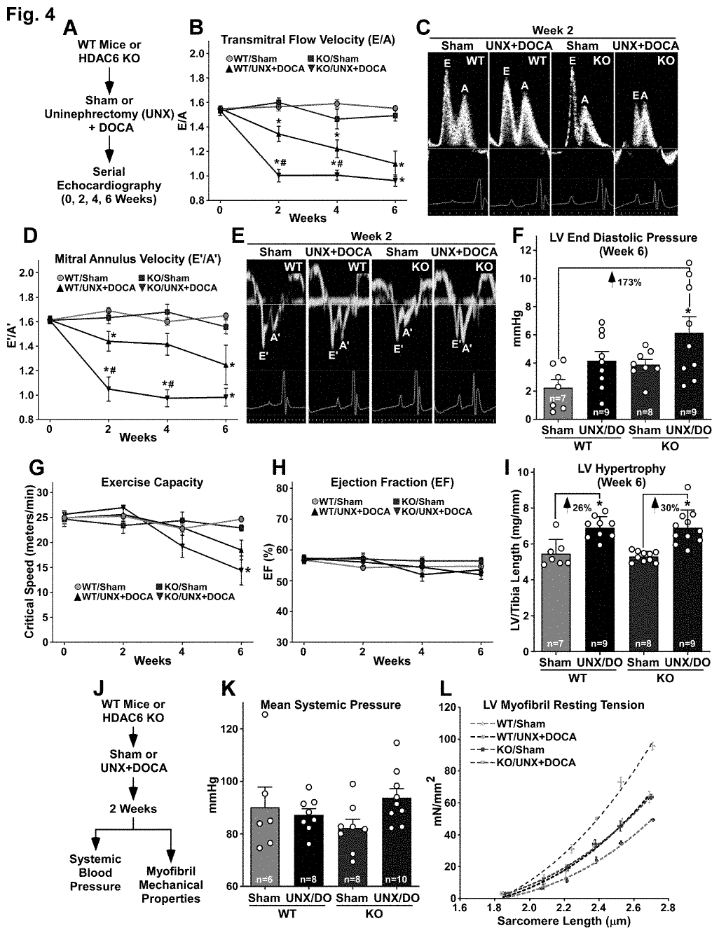

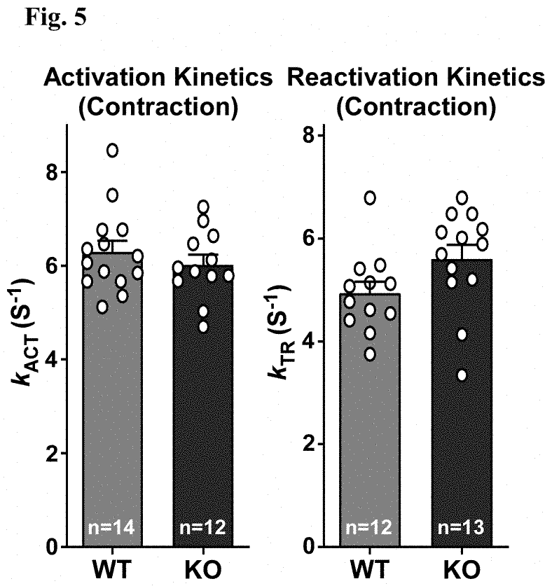

HDAC6 loss-of-function exacerbates diastolic dysfunction in a mouse model. Given that increased passive stiffness leads to impaired cardiac relaxation, we hypothesized that diastolic dysfunction would be exacerbated by HDAC6 deletion. To test this, HDAC6 KO mice and WT controls were evaluated in a model of hypertension-induced diastolic dysfunction with preserved ejection fraction driven by combined uninephrectomy (UNX) and deoxycorticosterone acetate (DOCA) (FIG. 4A). Serial Doppler echocardiography was used to quantify diastolic function by measuring the E/A ratio of the early filling (E) phase of the LV during diastole, which is due to relaxation of the LV, and the late filling (A) phase, which is mediated by contraction of the atrium. Two and four weeks post-UNX/DOCA, HDAC6 KO exhibited more severe diastolic dysfunction than WT controls, as evidenced by pronounced reduction in E/A (FIGS. 4B-4C). Doppler measurements of septal mitral annulus velocity (E′/A′) confirmed the more rapid onset of diastolic dysfunction in KO mice compared to WT controls (FIGS. 4D-4E). At the study endpoint of 6 weeks, E/A and E′/A′ were equivalent between KO and WT mice. Nonetheless, invasive hemodynamic measurements obtained at this time confirmed that KO mice subjected to UNX/DOCA had elevated LV end diastolic pressure than controls (FIG. 4F). Control and KO mice had preserved EF throughout the 6-week study (FIG. 4G).

Diastolic dysfunction is often attributed to cardiac hypertrophy and fibrosis (11, 26). However, KO and WT mice developed equivalent hypertrophy in response to UNX/DOCA, as determined by LV mass-to-tibia length measurements (FIG. 4H). Furthermore, Picroisirius red staining of LV sections failed to reveal significant interstitial fibrosis in any of the groups (FIG. 4I).

To further address the mechanism of more severe diastolic dysfunction in HDAC6 KO mice, a repeat study was performed, with analyses focusing on the 2-week time point when the difference in diastolic dysfunction between WT and KO mice is most exaggerated (FIG. 4J). Tail-cuff measurements revealed that mice subjected to UNX/DOCA remained normotensive 2 weeks post-surgery (FIG. 4K), demonstrating that the observed diastolic dysfunction at this early stage occurred independent of high blood pressure. In contrast, sarcomere length-to-resting tension curves revealed that UNX/DOCA treatment for 2 weeks led to stiffening of LV myofibrils, and the reduction in myofibril compliance was exaggerated in mice lacking HDAC6 (FIG. 4L). These data suggest that the intensified diastolic dysfunction in HDAC6 KO mice is due to increased stiffening of titin.

### REFERENCES

1. H. L. Granzier, T. C. Irving, Passive tension in cardiac muscle: contribution of collagen, titin, microtubules, and intermediate filaments. Biophys J68, 1027-1044 (1995).

2. W. A. Linke, Titin Gene and Protein Functions in Passive and Active Muscle. Annu Rev Physiol 80, 389-411 (2018).

3. A. Borbely et al., Hypophosphorylation of the Stiff N2B titin isoform raises cardiomyocyte resting tension in failing human myocardium. Circ Res 104, 780-786 (2009).

4. I. Makarenko et al., Passive stiffness changes caused by upregulation of compliant titin isoforms in human dilated cardiomyopathy hearts. Circ Res 95, 708-716 (2004).

5. S. F. Nagueh et al., Altered titin expression, myocardial stiffness, and left ventricular function in patients with dilated cardiomyopathy. Circulation 110, 155-162 (2004).

6. P. G. Vikhorev et al., Abnormal contractility in human heart myofibrils from patients with dilated cardiomyopathy due to mutations in TTN and contractile protein genes. Sci Rep 7, 14829 (2017).

7. J. S. Ware, S. A. Cook, Role of titin in cardiomyopathy: from DNA variants to patient stratification. Nat Rev Cardiol 15, 241-252 (2018).

8. A. E. Hopf et al., Diabetes-Induced Cardiomyocyte Passive Stiffening Is Caused by Impaired Insulin-Dependent Titin Modification and Can Be Modulated by Neuregulin-1. Circ Res 123, 342-355 (2018).

9. F. Koser, C. Loescher, W. A. Linke, Posttranslational modifications of titin from cardiac muscle: how, where, and what for? FEBS J, (2019).

10. L. van Heerebeek et al., Low myocardial protein kinase G activity in heart failure with preserved ejection fraction. Circulation 126, 830-839 (2012).

11. M. R. Zile et al., Myocardial stiffness in patients with heart failure and a preserved ejection fraction: contributions of collagen and titin. Circulation 131, 1247-1259 (2015).

12. O. Cazorla, Y. Wu, T. C. Irving, H. Granzier, Titin-based modulation of calcium sensitivity of active tension in mouse skinned cardiac myocytes. Circ Res 88, 1028-1035 (2001).

13. N. Fukuda, D. Sasaki, S. Ishiwata, S. Kurihara, Length dependence of tension generation in rat skinned cardiac muscle: role of titin in the Frank-Starling mechanism of the heart.

Circulation 104, 1639-1645 (2001).

14. W. A. Linke, N. Hamdani, Gigantic business: titin properties and function through thick and thin. Circ Res 114, 1052-1068 (2014).

15. S. Lahmers, Y. Wu, D. R. Call, S. Labeit, H. Granzier, Developmental control of titin isoform expression and passive stiffness in fetal and neonatal myocardium. Circ Res 94, 505-513 (2004).

16. C. A. Opitz, M. C. Leake, I. Makarenko, V. Benes, W. A. Linke, Developmentally regulated switching of titin size alters myofibrillar stiffness in the perinatal heart. Circ Res 94, 967-975 (2004).

17. C. M. Warren, P. R. Krzesinski, K. S. Campbell, R. L. Moss, M. L. Greaser, Titin isoform changes in rat myocardium during development. Mech Dev 121, 1301-1312 (2004).

18. W. Guo et al., RBM20, a gene for hereditary cardiomyopathy, regulates titin splicing. Nat Med 18, 766-773 (2012).

19. B. Pieske et al., Vericiguat in patients with worsening chronic heart failure and preserved ejection fraction: results of the SOluble guanylate Cyclase stimulatoR in heArT failurE patientS with PRESERVED EF (SOCRATES-PRESERVED) study. Eur Heart J 38, 1119-1127 (2017).

20. M. M. Redfield et al., Effect of phosphodiesterase-5 inhibition on exercise capacity and clinical status in heart failure with preserved ejection fraction: a randomized clinical trial. JAMA 309, 1268-1277 (2013).

21. M. Y. Jeong et al., Histone deacetylase activity governs diastolic dysfunction through a nongenomic mechanism. Sci Transl Med 10, (2018).

22. A. Matsuyama et al., In vivo destabilization of dynamic microtubules by HDAC6-mediated deacetylation. EMBO J 21, 6820-6831 (2002).

23. J. H. Kalin, J. A. Bergman, Development and therapeutic implications of selective histone deacetylase 6 inhibitors. J Med Chem 56, 6297-6313 (2013).

24. Y. Zhang, B. Gilquin, S. Khochbin, P. Matthias, Two catalytic domains are required for protein deacetylation. J Biol Chem 281, 2401-2404 (2006).

25. H. Zou, Y. Wu, M. Navre, B. C. Sang, Characterization of the two catalytic domains in histone deacetylase 6. Biochem Biophys Res Commun 341, 45-50 (2006).

26. S. F. Mohammed et al., Coronary microvascular rarefaction and myocardial fibrosis in heart failure with preserved ejection fraction. Circulation 131, 550-559 (2015).

1. Method for modulating cardiac muscle stiffness, comprising the step of administering a therapeutic amount of an HDAC6 inhibitor to a patient in need thereof

2. The method of claim 1, wherein the step of administering further comprises administering a HDAC6 inhibitor selected from the group of tubastatin A and givinostat.

3. The method of claim 1, further comprising the step of modulating titin stiffness.

4. The method of claim 3, further comprising the step of modulating myofibril resting tension.

5. The method of claim 1, further comprising the step of modulating one of active or passive cardiac muscle stiffness.

6. A method of treating dilated cardiomyopathy, comprising the step of administering a pharmacologically acceptable amount of an HDAC6 inhibitor.

7. The method of claim 6, wherein the step of administering further comprises administering a HDAC6 inhibitor selected from the group of tubastatin A and givinostat.

8. The method of claim 6, further comprising the step of modulating titin stiffness.

9. The method of claim 8, further comprising the step of modulating myofibril resting tension.

10. A method treating heart failure with a preserved ejection fraction, comprising the step of administering a pharmacologically acceptable amount of an HDAC6 activator.

11. The method of claim 10, further comprising the step of modulating titin stiffness.

12. The method of claim 11, further comprising the step of modulating myofibril resting tension.

13. A method of reducing myofibril resting tension comprising the step of administering a therapeutic amount of HDAC6.

14. The method of claim 13, wherein the step of administering a therapeutic amount of HDAC6 further comprises the step of administering recombinant HDAC6.

15. The method of claim 13, further comprising the step of administering protein kinase C (PKC) prior to the step of administering the therapeutic amount of HDAC6.

16. The method of claim 15, wherein the step of administering protein kinase C further comprises the step of administering recombinant protein kinase C.

17. A method for treating heart failure with preserved ejection fraction (HFpEF) comprising the step of selectively activating HDAC6 expression and reducing titin protein stiffness in cardiac muscle.

18. The method of claim 17, further comprising the step of administering to a patient in need thereof a pharmacologically effective amount of an HDAC6 activator.

19. The method of claim 18, wherein the HDAC6 activator further comprises a viral vector expressing wild type HDAC6.

20. The method of claim 19, further comprising the step of administering an enzyme harboring amino acid substitutions that abolish catalytic activity of HDAC6 selected from the group of Ad-HDAC6 H216A, Ad-HDAC6 H611A and combinations thereof.

21. An HDAC6 composition including one of an HDAC6 inhibitor or HDAC6 activator for use in a method for modulating cardiac muscle stiffness.

22. A containerized pharmaceutical product comprising a container containing a formulation comprising one of an HDAC6 inhibitor or an HDAC6 activator and a pharmaceutically acceptable carrier thereof, and instructions associated with said container directing use of the formulation in the treatment or prophylaxis of cardiac muscle stiffness.

23. A kit comprising at least one dosage units intended for at least one treatment period formulated in a form for delivery of one of an HDAC6 inhibitor, an HDAC6 activator or a pharmaceutically acceptable salt thereof, the dosage unit comprises a therapeutically effective amount of HDAC6 inhibitor for titan mediated cardiac muscle stiffness.
